(19) United States
(12) Patent Application Publication
(10) Pub. No.: US 2026/0097164 A1
(43) Pub. Date: Apr. 9, 2026

(54) NEGATIVE PRESSURE WOUND THERAPY DEVICE

(71) Applicants: Mohammad Shahani, Behshahr (IR); Nasser Ashgriz, Thornhill (CA)

(72) Inventors: Mohammad Shahani, Behshahr (IR); Nasser Ashgriz, Thornhill (CA)

(21) Appl. No.: 18/907,091

(22) Filed: Oct. 4, 2024

(51) Int. Cl. A61M 1/00

(52) U.S. Cl. CPC A61M 1/962; A61M 1/966

(57) ABSTRACT

The present invention is an integral negative pressure wound therapy device that can reduce pressure on the wound site. The device comprises a bandage and a pump assembly integrated into the bandage. The bandage is adhered to the skin by a medical adhesive and comprises a wound-facing surface defining a first cavity and a non-wound-facing surface defining a second cavity. The first cavity is configured to cover a wound site to form a sealed wound environment between the wound site and the bandage, and the pump is integrated into the second cavity configured to reduce the pressure inside the sealed wound environment, and the bandage is configured to maintain the reduced pressure within the sealed wound environment. The bandage defines a channel extending from the first cavity to the second cavity providing a fluid communication between the cavities.

### FIELD OF THE INVENTION

The present invention relates to a negative pressure wound device for providing negative pressure to wound sites to promote wound healing.

### BACKGROUND OF THE INVENTION

Negative pressure wound therapy (NPWT) is a well-established medical procedure used to promote wound healing. It involves applying controlled sub-atmospheric pressure to a wound site, typically through a dressing and drainage tube connected to a pump. NPWT offers several benefits for wound healing, including increased blood flow to the wound site to help deliver oxygen and nutrients and different kinds of cells necessary for tissue repair, reduced edema (swelling) to improve blood flow, and reduced pain, promotion of new tissue to fill the wound bed during the healing process, and reduction of bacteria and debris from the wound. Traditionally, NPWT devices have relied on bulky and cumbersome electric pumps. These pumps require a power source and can be inconvenient for patients, especially those who are mobile or require frequent dressing changes.

### SUMMARY OF THE INVENTION

An integral negative pressure wound therapy device is disclosed that can reduce pressure on the wound site. The device comprises a bandage and a pump assembly integrated into the bandage. The bandage is adhered to the skin by a medical adhesive. The bandage comprises a wound-facing surface defining a first cavity and a non-wound-facing surface defining a second cavity. The first cavity is configured to cover a wound site to form a sealed wound environment between the wound site and the bandage. The pump is integrated into the second cavity configured to reduce the pressure inside the sealed wound environment. The bandage is configured to maintain the reduced pressure within the sealed wound environment. The bandage defines a channel extending from the first cavity to the second cavity providing a fluid communication between the cavities.

In one embodiment, the pump is a peristaltic pump comprising a tube, a rotational mechanism, and a lock-unlock mechanism, all placed in a housing. The first opening of the tube is connected to the channel and has fluid communication with the sealed wound environment and the second opening of the tube is open and has fluid communication with the outside environment. A string is provided that has its one end attached to the rotational mechanism and wrapped around the rotational mechanism. When pulled, the string initiates the rotation of the rotational mechanism. The rotational mechanism is configured to reduce pressure in the wound environment by transmitting its rotational force to the tube, isolating a volume of wound environment air within the tube, and evacuating the isolated air volume to the outside environment during rotation in a first direction upon pulling the string in a lock state. The rotational mechanism is further configured to retract the string during rotation in a second direction opposite the first direction upon releasing the string in an unlock state.

The lock-unlock mechanism is connected to the rotational mechanism configured to selectively enable or disable the transmission of the rotational force of the rotational mechanism to the tube in the lock state and unlock state respectively. When the lock-unlock mechanism enables the transmission of the rotational force to the tube in the lock state, by pulling the string the rotational mechanism isolates the volume of air in the tube and evacuates it from the wound environment to the outside environment. When the lock-unlock mechanism disables the transmission of the rotational force to the tube in the unlock state, by releasing the string the rotational mechanism will not isolate the volume of the air in the tube and evacuate it from the outside environment to the wound environment.

A one-way valve is positioned in the channel configured to provide a one-way fluid communication from the wound environment to the tube.

### BRIEF DESCRIPTION OF THE DRAWINGS

Embodiments herein will hereinafter be described in conjunction with the appended drawings provided to illustrate and not to limit the scope of the claims, wherein like designations denote like elements, and in which:

FIG. 1A shows an isometric view of the embodiment of the invention;

FIG. 1B shows a top view of the embodiment of the invention;

FIG. 2 shows an exploded view of the embodiment of the invention;

FIG. 3A shows an isometric top view of the bandage of the invention;

FIG. 3B shows an isometric bottom view of the bandage of the invention;

FIG. 4 shows a section view of the bandage of the invention;

FIG. 5 shows a schematic cross-sectional view of the embodiment of the invention over a wound site;

FIG. 6A shows an assembly view of the rotational mechanism of the pump in one embodiment of the invention;

FIG. 6B shows an exploded view of the rotational mechanism of the pump in one embodiment of the invention;

FIG. 7A shows an upper view of the housing of the pump in the embodiment of the invention;

FIG. 7B shows a side view of the housing of the pump in the embodiment of the invention;

FIG. 7C shows a side cross-section view of the housing of the pump in the embodiment of the invention;

FIG. 8A shows a bottom view of the housing of the pump with a tube in the embodiment of the invention;

FIG. 8B shows an isometric bottom view of the housing of the pump with a tube in the embodiment of the invention;

FIG. 8C shows a bottom view of the housing of the pump with tube and rotor in the embodiment of the invention;

FIG. 9A shows the pump in the unlock state;

FIG. 9B shows the pump in the unlock state being pushed with an external force;

FIG. 9C shows the pump in the lock state;

FIG. 10 shows the pump in the unlock state while the string is pulled;

FIG. 11A shows the bottom view of the pump;

FIG. 11B shows another bottom view of the pump;

FIG. 11C shows another bottom view of the pump, and

FIG. 12 shows one embodiment of the invention in use.

### DETAILED DESCRIPTION

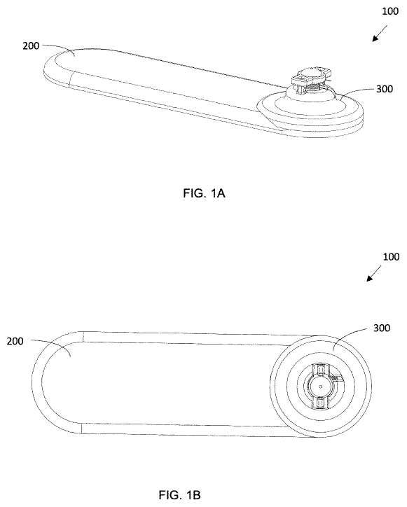

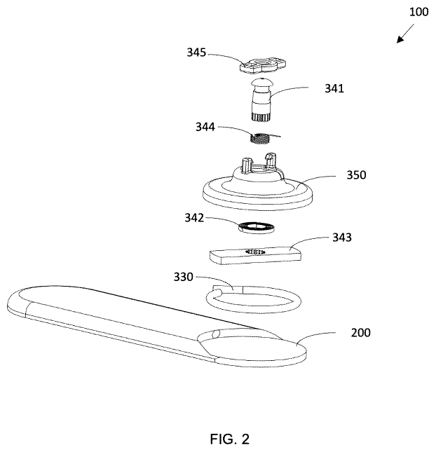

FIGS. 1A, 1B, and 2 show an isometric, a top, and an exploded view of one embodiment of the presently disclosed negative pressure wound therapy device 100, respectively. Device 100 comprises a bandage 200 configured to be placed over the wound site to form a sealed wound environment, and a pump 300 integrated into the bandage 200 to create a negative pressure on the wound bed by reducing the pressure in the wound environment. The bandage 200 is made of a flexible and resilient material, such as rubber or silicone that has contact with a dressing that covers the wound and is capable of maintaining reduced pressure underneath the bandage. The negative pressure and reduced pressure described herein are pressures below atmospheric pressure. The bandage 200 can be made in a variety of shapes and sizes to cover a variety of wound sites.

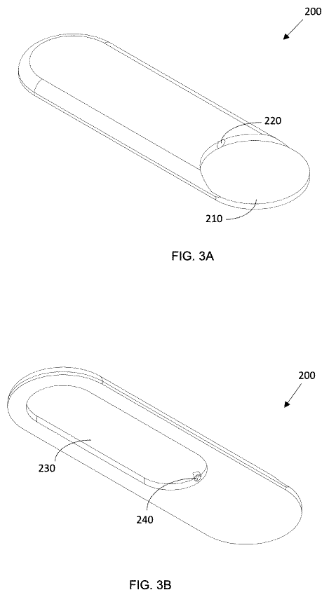

FIGS. 3A and 3B show a top and a bottom view of the bandage 200 respectively. The bandage comprises two surfaces: a wound-facing surface (FIG. 3B) and a non-wound-facing surface (FIG. 3A). In one embodiment, the bandage 200 is a round ended rectangular shaped surface, that has a first cavity 230 that is another smaller round ended rectangular shape cavity in the bandage 200 on the wound-facing surface. This surface is placed over the wound site to form a sealed wound environment. A second cavity 210 on the other end of the bandage on the non-wound-facing surface is provided to house the pump 300. A channel 220 between the first cavity 230 and the second cavity 210 is provided to house a one-way valve 240 enabling one-way fluid communication between cavities 210 and 230.

The pump may be detachable allowing the user to remove and reattach the pump from the bandage.

The wound-facing surface may have a non-slip coating, configured to enhance the device's ability to adhere securely to the wound site, minimizing the risk of displacement or dislodgement during treatment.

The bandage can be configured in various shapes, including but not limited to rectangular, circular, and oval shapes, and different sizes, thereby the device is adaptable for a wide range of wound types with various sizes and locations.

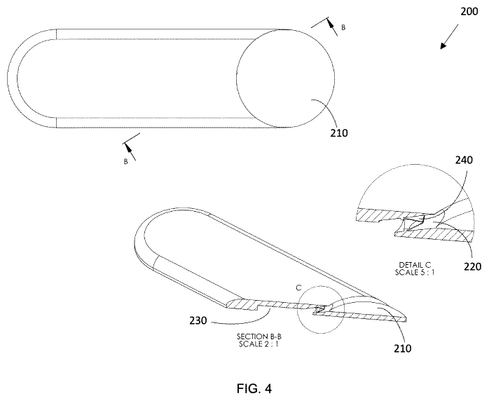

FIG. 4 shows a cross-sectional view of the bandage 200. The one-way valve 240 housed in the channel 220 enables one-way fluid transfer from the first cavity 230 into the second cavity 210 and prevents any fluid communication in the reverse direction.

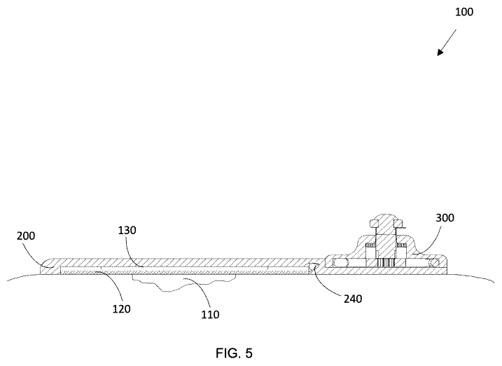

FIG. 5 shows the device 100 adhered to skin by a medical adhesive. The first cavity 230 covers the wound bed 110 and the wound dressing 120 forms a sealed wound environment 130 defined between the bandage 200, the wound bed 110, and the wound dressing 120. The pump 300 is configured to reduce the pressure inside the sealed wound environment 130 by evacuating a volume of air from the sealed wound environment 130 to the outside environment. The pump 300 is integrated into the bandage 200 on the second cavity 210 and is connected to the channel 220; thereby the pump 300 has one-way fluid communication with the wound environment 130 through the one-way valve 240 housed in the channel 220, wherein the one-way valve 240 enables fluid transfer from the wound environment 130 into the pump 300. The pump 300 evacuates a volume of the air inside the wound environment 130 to the outside environment through the channel 220 and the one-way valve 240. The one-way valve 240 prevents the air from returning to the sealed wound environment 130.

The channel may have a non-stick coating covering its walls. This coating minimizes the adhesion of fluids, such as wound exudate, to the channel walls, thereby preventing fluid accumulation and blockage.

The device may further have an absorbing layer integrated into the first cavity. This absorbing layer is designed to absorb exudate from the wound site, contributing to a cleaner and more controlled wound environment.

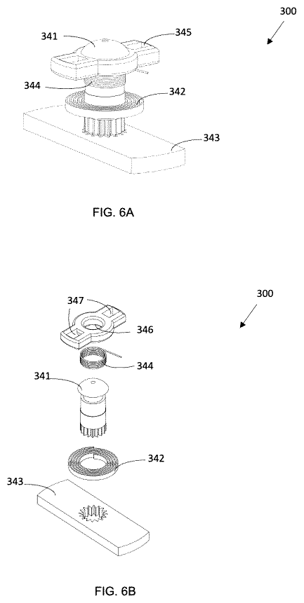

FIGS. 6A-B, 7A-C, 8A-C, 9A-C, 10, and 11A-C show one embodiment of the pump 300 utilized in the present disclosed invention. The pump 300 is a new peristaltic pump. It comprises a housing 350, a rotational mechanism 340, a lock-unlock mechanism, and a tube 330.

FIGS. 6A and 6B show an assembly and an exploded view of the rotational mechanism 340, respectively. The rotational mechanism comprises a shaft 341, a flat coil spring 342, a rotor 343, and a string 344. The shaft 341 is configured for both vertical and rotational motion relative to the bandage. The shaft 341 and rotor 343 are configured to connect temporarily using a splined coupling. The shaft 341 features an external spline design at one end and the rotor 343 features a corresponding internal spline design at its center. This configuration facilitates the transfer of rotational motion from the shaft 341 to the rotor 343. Upon disconnection of the coupling, the rotor 343 will no longer follow the rotational motion of the shaft 341.

The click part 345 is a part of the lock-unlock mechanism and defines three aligned holes through its structure: one in the center 346 and two on either side 347. The non-external spline designed end of the shaft 341 is connected to the click part 345 via the center hole 346. This configuration allows the click part 345 to follow the vertical motion of the shaft 341.

The flat coil spring 342 is coiled around the shaft 341 with its inner end connected to the shaft 341 and the outer end connected to housing 350. The string 344 is wrapped around the shaft 341, with one end connected to the shaft 341 and another end remaining free. When the string is not pulled, the flat coil spring 342 is in the rest state which means it is not under any force. By pulling the free end of the string 344, the pulling motion of the string 344 is converted into a rotational motion of the shaft 341. The inner end of the flat coil spring 342 rotates along with the shaft 341 while the outer end is connected to the housing 350 and is fixed, therefore a torsional elastic potential energy is stored in the spring 342. Upon releasing the string 344, the respective torsional elastic potential energy stored in the spring 342 forces the spring 342 to get back to its rest state, therefore, the shaft 341 which is connected to the inner end of the flat coil spring 342 rotates along with the flat coil spring 342 and retract the string 344.

The rotational mechanism of the device may further have a damping mechanism configured to dampen retraction speed of the string. This damping mechanism ensures a smooth and controlled retraction process, preventing potential damage to the wound or surrounding tissues due to excessive force.

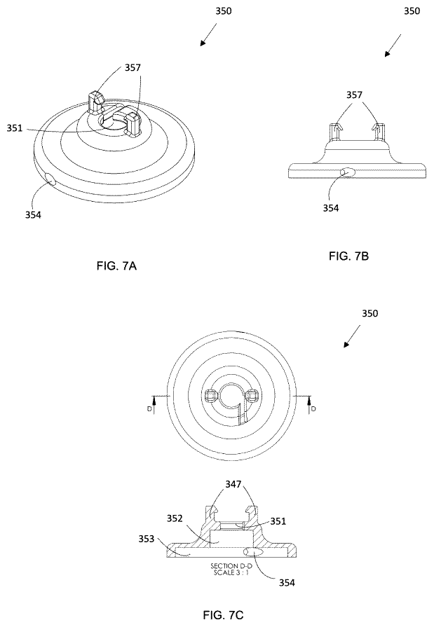

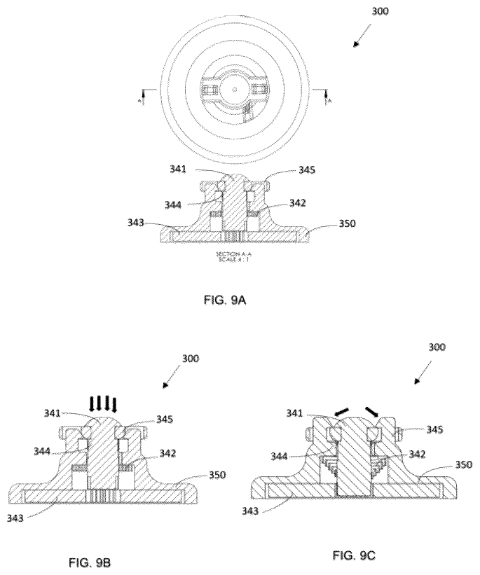

FIGS. 7A-C and 8A-C show the embodiment of the housing 350 of the pump 300 utilized in the present disclosed device 100. The housing 350 is configured to house the rotational mechanism 340, the lock-unlock mechanism, and the tube 330. The housing 350 has a bell-shaped body defining an opening 351 at the top. The housing 350 has a pair of knobs 357 with the same radius, positioned on the top surface and centered on opposite sides of the opening 351, wherein the knobs 357 are a part of the lock-unlock mechanism. The interior cavity of the housing 350 is divided into two spaces of different sizes: an upper space 352 and a lower space 353. The upper space 352 houses the flat coil spring 342 and the lower space 353 houses the rotor 343 and the tube 330. The shaft 341 passes through the housing 350 and the flat coil spring 342. (FIG. 9A) The housing 350 defines a hole 354 in its structure in the lower space 353 that is the same size as the opening of the channel 220 in the bandage 200.

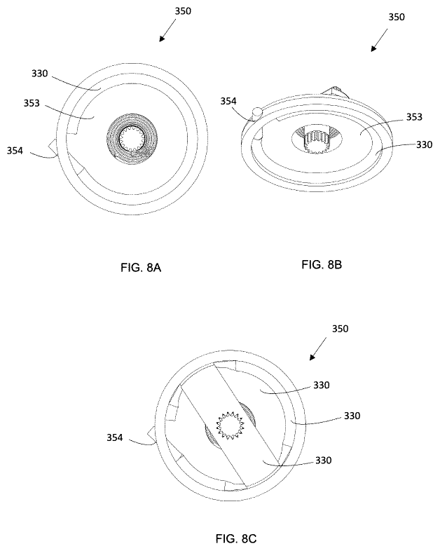

As shown in FIGS. 8A and 8B the tube 330 is positioned along the inner perimeter of the lower space 353 of the housing 350. The tube 330 has a first opening connected to the channel 220 providing fluid communication between the tube and the sealed wound environment and a second opening having fluid communication with the outside environment. In this configuration, the sealed wound environment has a one-way fluid communication with the outside environment through the tube 330. The rotor 343 is positioned in the lower space 353 and has two wings in contact with the tube 330. The rotor 343 is configured to push the tube 330 against the inner wall of the housing 350 with its wings. The tube 330 is compressed at the contacting points between the wings and the tube 330, thereby a volume of air is trapped in the tube 330 between the two compressed points (FIG. 8C).

FIGS. 9A-C show the cross-sectional view of the pump 300 and the lock-unlock mechanism. The lock-unlock mechanism comprises the pair of knobs 357 and the click part 345 and is configured to manually change the state of the pump between lock state and unlock state. The side holes 347 of the click part 345 are configured to lock the knobs 357 inside. In FIG. 9A the pump 300 is in the unlock state and the string 344 is wrapped around the shaft 341. In the unlock state, shaft 341 is free to move vertically and is not connected with the rotor 343, and the flat coil spring 342 is in the rest state which means it is not under any force. By pushing the top of the shaft 341 with an external force like a finger the state of the pump 300 can be changed from the unlock state to the lock state (FIG. 9B [symbol] . FIG. 9C shows the pump 300 in the lock state wherein the shaft 341 is pushed until the click part 345 sticks under the knobs 357. In the lock state, the shaft 341 is connected to the rotor 343, and the inner end of the flat coil spring 342 which is connected to the shaft 341 is moved with the shaft 341 while the outer end is connected to the housing 350 and is fixed, therefore an axial elastic potential energy is stored in the spring 342. The knobs 357 are designed to be pushed outwards from each other with an applied external force to change the state of pump 300 from the lock state to the unlock state. Upon pushing the knobs 357 outwards from each other, the klick part 345 is released from the knobs 357 and the shaft 341 is free to move vertically. The respective axial elastic potential energy stored in the spring 342 forces the spring 342 to get back to its rest state therefore the shaft 341 which is connected to the inner end of the flat coil spring 342 moves along with the flat coil spring 342 and disconnects from the rotor 343.

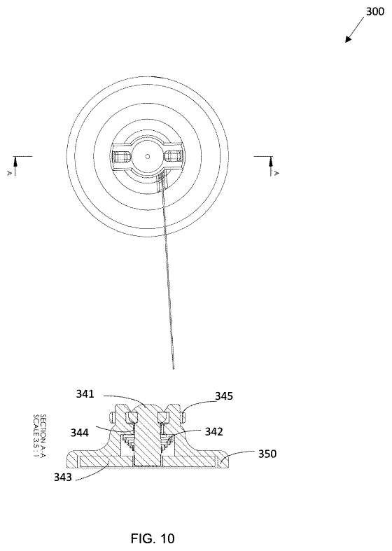

FIG. 10 shows the pump in the lock state when the free end of the string 344 is pulled. By pulling the free end of the string 344, the pulling motion of the string 344 is converted into a rotational motion of the shaft 341, and the inner end of the flat coil spring 342 rotates along with the shaft 341 while the outer end is connected to the housing 350 and is fixed, therefore a torsional elastic potential energy is stored in the spring 342.

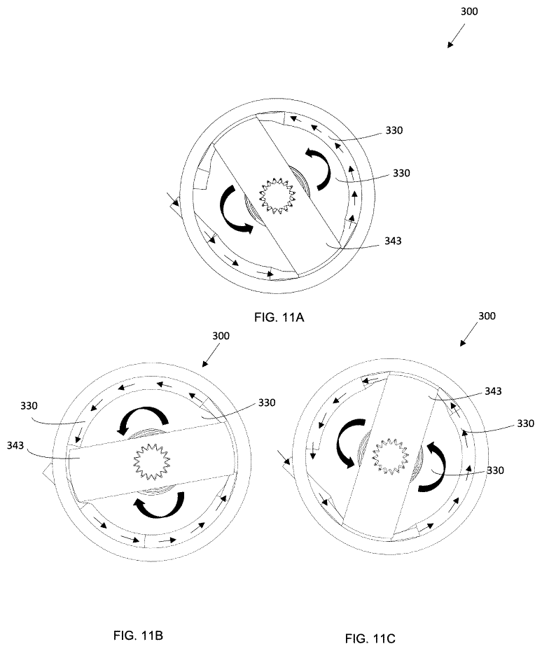

FIGS. 11A-C show the bottom view of the pump 300 in the lock state while the string 344 is being pulled. The rotor traps a volume of air in the tube and isolates it between the compressed point of the tube 330. As the rotor 343 rotates in the direction of the shaft 341, the wings move along the tube 330 shifting the location of the compressed points from the first opening to the second opening of the tube 330, thereby the volume of air trapped in the tube 330 is moved through the tube 330 and the tube 330 is left empty of air behind. The empty tube 330 is in one-way fluid communication with the wound environment through its first opening connected to the channel 220. The empty tube 330 has lower pressure than in the wound environment, therefore a volume of air from the wound environment goes to the empty tube 330 (FIG. 11A). When one of the wings passes the tube's second opening 330, the trapped air can access the outside environment. The other wing pushes the trapped air along the tube 330 until it goes into the outside environment at the tube's second opening 330 (FIG. 11B). When one of the wings passes the tube's first opening 330, the air coming from the sealed wound environment is again trapped between two wings and forced to move inside the tube 330 until it goes to the outside environment at the tube's second opening 330 (FIG. 11C). In each cycle of the rotor's rotation, a volume of the air inside the sealed wound environment is evacuated through the tube 330, reducing pressure inside the sealed wound environment until the pressure gets in the aimed range.

The string 344 can be pulled several times until the pressure of the sealed wound environment gets in an aimed range. When the string 344 is fully pulled, the flat coil spring 342 should get back to the rest state. By unlocking the pump, the axial elastic potential energy stored in the spring transmits to the vertical motion of the shaft, therefore, the shaft disconnects from the rotor. By releasing the string 344 in the unlock state, the torsional elastic potential energy stored in the spring 342 transmits to the rotational motion of the shaft 341, therefore, the shaft 341 retracts the string 344. By releasing the string 344 in the unlock state, where the shaft 341 and rotor 343 are disconnected, the device ensures that the rotor 343 is not rotating with the shaft 341 and won't transmit air from the outside environment to the sealed wound environment.

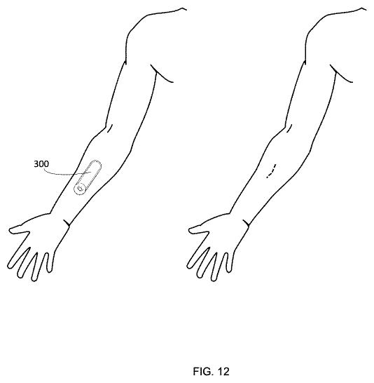

FIG. 12 shows one embodiment of the invention in use, where the device is positioned on the wound site. The bandage is adhered to skin using medical adhesive.

The bandage may be made of transparent material allowing user to have direct visibility of the wound healing progress, and to monitor the wound condition without removing the bandage.

The integral negative pressure wound therapy device may further comprise an electronic pressure sensor with a display or an indicator. The pressure sensor may be integrated into the bandage and use a battery as a power source, monitoring the pressure within the sealed wound environment and displaying the pressure level. The pressure sensors may provide feedback to the user upon reduction in negative pressure obtained within the sealed wound environment.

The presently disclosed integral negative pressure wound therapy device may further having a manual valve positioned within the channel. This valve is configured to close the channel when manually operated by the user, preventing any fluid communication between the wound environment and the outside environment.

The integral negative pressure wound therapy device may further having a ratchet mechanism comprising a ratchet wheel and a pawl. The ratchet wheel has teeth on its outer perimeter and defines a hole through its center. The shaft passes through the respective hole. The ratchet wheel is fixed to the shaft, thereby the ratchet wheel replicates both vertical and rotational motion of the shaft. The pawl is connected to the housing from one end and is configured to engage and disengage with the teeth of the ratchet wheel from the other end in lock and unlock states, respectively. The ratchet mechanism is configured to permit the rotation of the rotor in the first direction and to prevent the rotation of the rotor in the second direction in the lock state when the pawl is engaged with the ratchet wheel. Thereby, in the lock state, when the string is pulled, the ratchet mechanism allows the rotor to rotate in the first direction and evacuate a volume of air from the wound environment to the outside environment, and when the string is released the ratchet mechanism prevents the rotor to rotate in the second direction and transfer a volume of air from the outside environment to the wound environment.

The integral negative pressure wound therapy device may further having a pressure-limiting bottleneck design on the first opening of the tube. The pressure-limiting bottleneck design is configured to start collapsing and reducing the diameter of the first opening of the tube when the negative pressure inside the wound environment is within an aimed pressure range. The aimed pressure range is between 60 mm Hg and about 180 mm Hg. When the reduced pressure inside the wound environment exceeds the aimed pressure range, the pressure-limiting bottleneck design closes the first opening and the channel and prevents any fluid communication between the wound environment and the outside environment. The pressure-limiting bottleneck design on the first opening of the tube ensures that the negative pressure inside the wound environment never exceeds the aimed pressure range.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

With respect to the above description, it is to be realized that the optimum relationships for the parts of the invention in regard to size, shape, form, materials, function, and manner of operation, assembly, and use are deemed readily apparent and obvious to those skilled in the art, and all equivalent relationships to those illustrated in the drawings and described in the specification are intended to be encompassed by the present invention.

1) An integral negative pressure wound therapy device, comprising:
a) a bandage, comprising:
i) a wound-facing surface defining a first cavity configured to cover a wound site to form a sealed wound environment between the wound site and the bandage;
ii) a non-wound-facing surface defining a second cavity, and
iii) a channel extending from the first cavity to the second cavity;
b) a pump assembly integrated into the second cavity, and configured to reduce pressure in the sealed wound environment, comprising:
i) a flexible tube having a first opening connected to the channel, configured to communicate fluidly with the sealed wound environment, and a second opening configured to communicate fluidly with an ambient environment;
ii) a rotational mechanism having a string wound around the rotational mechanism, with one end of the string attached to the rotational mechanism, wherein the string configured to rotate the rotational mechanism upon being pulled, and wherein the rotational mechanism is configured to:
reduce pressure in the sealed wound environment by transmitting a rotational force to the flexible tube, isolating a volume of a sealed wound environment air within the flexible tube, and evacuating said volume to ambient environment during rotation in a first direction upon pulling the string in a lock state; and
retract the string during rotation in a second direction opposite the first direction upon releasing the string in an unlock state;
iii) a lock-unlock mechanism connected to the rotational mechanism configured to selectively enable or disable the transmission of the rotational force of the rotational mechanism to the flexible tube in the lock state and the unlock state, respectively, thereby enabling air isolation and evacuation from the sealed wound environment to the ambient environment upon pulling the string and disabling air isolation and evacuation from the ambient environment to the sealed wound environment upon releasing the string, and
iv) a housing to accommodate the rotational mechanism, the lock-unlock mechanism, and the tube.

2) The integral negative pressure wound therapy device of claim 1, wherein the bandage further comprises a one-way valve positioned in the channel configured to provide one-way fluid communication from the sealed wound environment to the flexible tube.

3) The integral negative pressure wound therapy device of claim 1, wherein the rotational mechanism comprises a shaft, a flat coil spring coiled around the shaft, a rotor, and said string, wherein the shaft is configured for both vertical and rotational motion relative to the bandage, and wherein the rotor and the shaft have a splined coupling, thereby having a temporary connection, and wherein the rotor transmits the rotational force of the rotational mechanism to the flexible tube, and wherein the flat coil spring having one end attached to the shaft and the other end attached to the housing, the flat coil spring is configured to move the shaft vertically away from the bandage, thereby disconnecting the shaft and the rotor in the unlock state, and wherein the string wounds around the shaft, with one end of the string attached to the shaft, and the string configured to rotate the rotational mechanism upon being pulled.

4) The integral negative pressure wound therapy device of claim 3, wherein the lock-unlock mechanism comprises a click part connected to the shaft and a pair of knobs positioned on the housing, wherein the click part and the knobs are configured to lock together and unlock from each other manually, and wherein in the lock state the rotor is connected to the shaft and in the unlock state the rotor is disconnected from the shaft, whereby the lock state is activated by locking the click part with the knobs and the unlock state is activated by unlocking the click part from the knobs.

5) The integral negative pressure wound therapy device of claim 1, wherein the bandage is made of a flexible and resilient material.

6) The integral negative pressure wound therapy device of claim 1, wherein the bandage having a medical adhesive to adhere to skin.

7) The integral negative pressure wound therapy device of claim 1, wherein the channel further having a non-stick coating to reduce adhesion of fluids to channel walls, thereby preventing fluid accumulation and blockage.

8) The integral negative pressure wound therapy device of claim 1, wherein the rotational mechanism further having a damping mechanism, configured to dampen retraction speed of the string.

9) The integral negative pressure wound therapy device of claim 3, further having a ratchet mechanism comprising:
a) a ratchet wheel defining a hole through it which the shaft passes and is fixed to the shaft, thereby the ratchet wheel replicates both vertical and rotational motion of the shaft, and
b) a pawl connected to the housing from one end, configured to permit and prevent the rotation of the ratchet wheel and the shaft in the first and second direction, respectively, upon engaging with the teeth of the ratchet wheel with its other end,
whereby the pawl engages and disengages with the ratchet wheel in the lock state and the unlock state, respectively.

10. The integral negative pressure wound therapy device of claim 1, further having an absorbing layer integrated into the first cavity to absorb exudate from the wound site.

11. The integral negative pressure wound therapy device of claim 1, further having an electronic pressure sensor powered by a disposable battery, wherein the pressure sensor is integrated into the bandage to monitor the pressure within the sealed wound environment, and wherein the pressure sensor comprises a display or an indicator to display or indicate the pressure level within the sealed wound environment measured by the pressure sensor.

12. The integral negative pressure wound therapy device of claim 1, wherein the first opening of the tube having a pressure-limiting bottleneck design configured to reduce diameter of the first opening upon the reduced pressure inside the wound environment increasing within a predefined pressure range, and to collapse upon the reduced pressure inside the wound environment exceeding the predefined pressure range, thereby closing the channel and preventing any fluid communication between the wound environment and the ambient environment when the reduced pressure inside the wound environment exceeds the predefined pressure range.

13. The integral negative pressure wound therapy device of claim 12, wherein the predefined pressure range is between 60 mm Hg and about 180 mm Hg.

14. The integral negative pressure wound therapy device of claim 1, wherein the pump is detachable.

15. The integral negative pressure wound therapy device of claim 1, wherein the wound-facing surface having a non-slip coating.

16. The integral negative pressure wound therapy device of claim 1, further having a manual valve positioned in the channel configured to close the channel, thereby preventing any fluid communication between the wound environment and the ambient environment.

17. The integral negative pressure wound therapy device of claim 1, wherein at least a portion of the bandage is transparent, thereby allowing for visibility of the wound healing progress.

18. The integral negative pressure wound therapy device of claim 1, wherein the bandage is rectangular, circular or oval.
